(19) United States
(12) Patent Application Publication
(10) Pub. No.: US 2017/0158645 A1
(43) Pub. Date: Jun. 8, 2017

(54) AMORPHOUS FORM AND NEW CRYSTALLINE FORMS OF MACITENTAN

(71) Applicant: OLON S.p.A., Rodano (MI) (IT)

(72) Inventors: Giorgio BERTOLINI, Rodano (MI) (IT); Lazzaro FELICIANI, Rodano (MI) (IT); Ilaria FERRANDO, Rodano (MI) (IT)

(21) Appl. No.: 15/325,907

(22) PCT Filed: Jul. 13, 2015
(86) PCT No.: PCT/IB2015/055281

§ 371 Date: Jan. 12, 2017

(30) Foreign Application Priority Data
Jul. 15, 2014 (IT) MI2014A001283

(51) Int. Cl. C07D 239/47

(52) U.S. Cl. CPC C07D 239/47; C07B 2200/13

(57) ABSTRACT

The present invention relates to the amorphous form of macitentan and to new crystalline forms thereof. The invention also relates to processes for the preparation of the new compounds, to the pharmaceutical compositions comprising them and to the use thereof in the therapy.

### SUMMARY OF THE INVENTION

The present invention relates to the amorphous form of macitentan and new crystalline forms thereof. The invention also relates to processes for the preparation of the new compounds, the pharmaceutical compositions comprising them and the use thereof in the therapy.

### TECHNICAL FIELD

Macitentan is the International Common Denomination (ICD) of the compound N-[5-(4-bromophenyl)-6-[2-[(5-bromo-2-pyrimidinyl)oxy]ethoxy]-4-pyrimidinyl]-N′-propyl-sulfamide of formula (I)

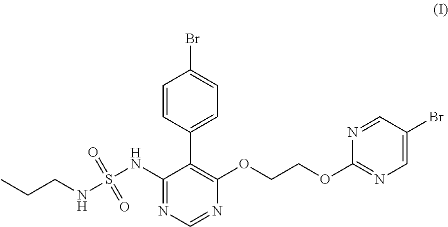 [SMILES: CCCNS(=O)(=O)Nc1ncnc(OCCOc2ncc(Br)cn2)c1-c1ccc(Br)cc1]

Macitentan is an endothelin receptor antagonist compound active in the oral form and has been recently introduced in the therapy for the treatment of pulmonary arterial hypertension, alone or in combination with other active ingredients.

J. Med. Chem. 2012, 55, 7849-7861 describes two crystalline forms of macitentan, obtained by re-crystallizing macitentan from ethyl acetate/hexane mixture and methanol, respectively. In particular, one of the two polymorphs, defined as form I, is pure whereas the polymorph, defined as form II, is a methanol solvate.

It is known that crystalline forms of active pharmaceutical ingredients can show different physico-chemical properties and can offer advantages, for example in terms of solubility, stability and bioavailability. As a consequence, the research and discovery of new crystalline forms of active pharmaceutical ingredients can result in more reliable and effective therapies.

For this reason, it is considered a contribution to the art the preparation of new crystalline forms of active pharmaceutical ingredients, because such new forms can allow to improve the stability, the bioavailability and the pharmacokinetics, to limit the hygroscopicity, and/or to aid the galenic and industrial manufacturing of the active pharmaceutical ingredients.

However, the preparation of such new crystalline forms is absolutely not evident, it is not predictable and not always possible.

Also for macitentan, there is an interest in looking for new crystalline forms showing physico-chemical properties suitable for a safe and effective therapeutic use.

It is also of significant interest the research of the amorphous forms of the pharmaceutical compounds, i.e. the non-crystalline forms, because it is known that an amorphous form of an active ingredient could be more bioavailable with respect to a crystalline form.

### OBJECTS OF THE INVENTION

It is an object of the invention to provide an amorphous (non-crystalline) form of macitentan.

It is another object of the invention to provide new crystalline forms of macitentan.

Another object of the invention is to provide processes for the preparation of said new compounds, of the pharmaceutical compositions comprising them and for the use thereof in the therapy.

### BRIEF DESCRIPTION OF THE DRAWINGS

FIG. 1 shows XRPD of the amorphous form of macitentan

FIG. 2 shows FT-IR spectrum of the amorphous form of macitentan

FIG. 3 shows DSC spectrum of the amorphous form of macitentan

FIG. 4 shows TGA spectrum of the amorphous form of macitentan

FIG. 5 shows XRPD of crystalline form III of macitentan

FIG. 6 shows FT-IR spectrum of crystalline form III of macitentan

FIG. 7 shows DSC spectrum of crystalline form III of macitentan

FIG. 8 shows TGA spectrum of crystalline form III of macitentan

FIG. 9 shows EGA spectrum of crystalline form III of macitentan

FIG. 10 shows XRPD of crystalline form IV of macitentan

FIG. 11 shows FT-IR spectrum of crystalline form IV of macitentan

FIG. 12 shows DSC spectrum of crystalline form IV of macitentan

FIG. 13 shows TGA spectrum of form IV of macitentan

FIG. 14 shows EGA spectrum of form IV of macitentan

FIG. 15 shows XRPD of crystalline form V of macitentan

FIG. 16 shows FT-IR spectrum of crystalline form V of macitentan

FIG. 17 shows DSC spectrum of crystalline form V of macitentan

FIG. 18 shows TGA spectrum of form V of macitentan

FIG. 19 shows EGA spectrum of form V of macitentan

### DESCRIPTION OF THE INVENTION

It has now been found that it is possible to obtain the amorphous form and new crystalline forms of macitentan.

The amorphous form and the new crystalline forms of macitentan of the invention have never been disclosed in the current literature and represent a subject-matter of the present invention.

As a consequence, a subject-matter of the invention, according to one of the aspects thereof, is an amorphous form, i.e. non-crystalline, of macitentan.

Another subject-matter of the invention, according to another of the aspects thereof, is a process for the preparation of the amorphous form of macitentan which comprises dissolving macitentan in dimethyl sulfoxide (DMSO) and then evaporating the solvent, preferably at a temperature between 30° C. and 80° C., advantageously around 60° C. The amorphous form of macitentan obtained and/or obtainable with the process described above, represents a further subject-matter of the present invention.

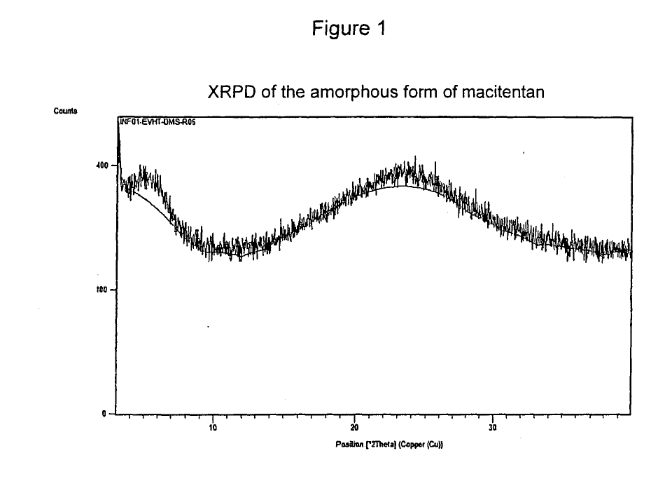

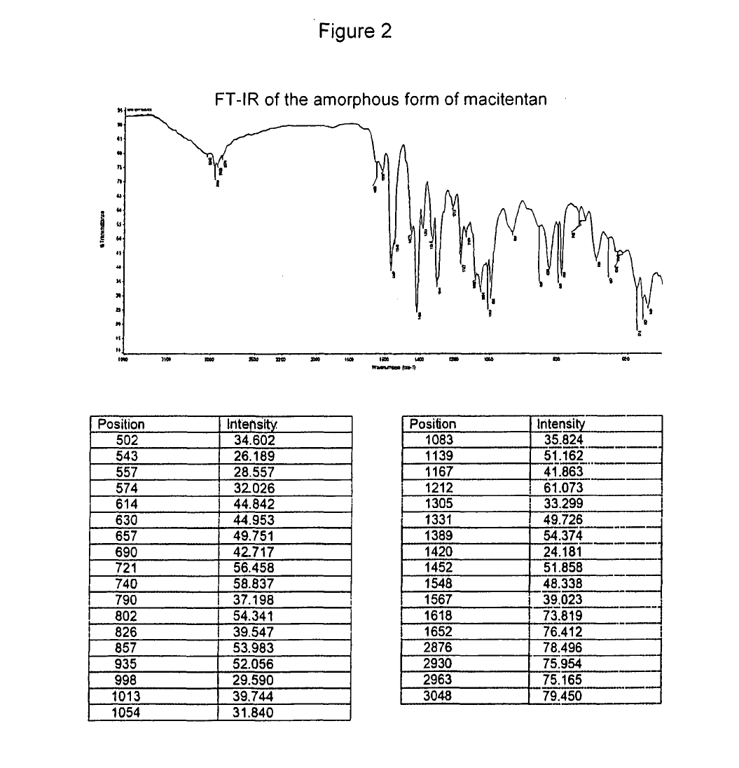

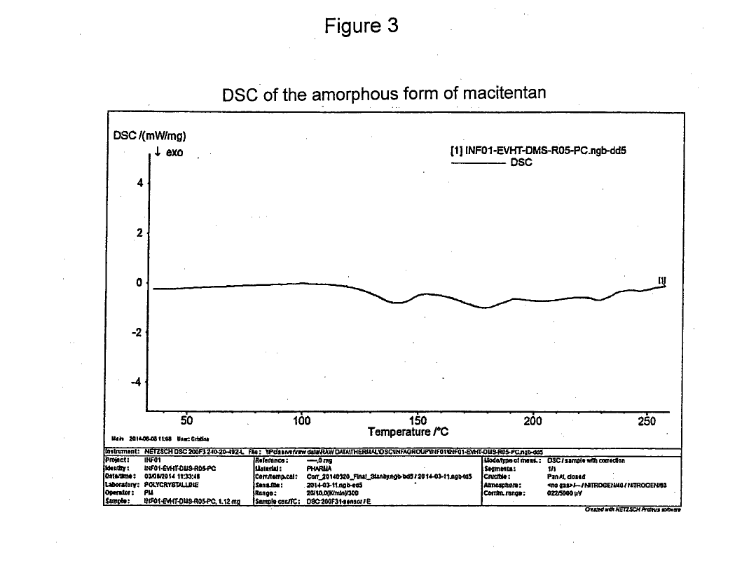

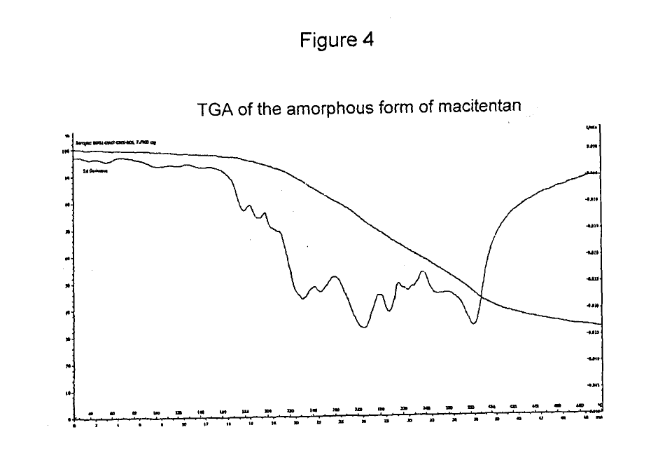

The amorphous form of macitentan has been characterized and the X-ray diffraction (XRPD), infrared (FT-IR), differential scanning calorimetry (DSC) spectra are provided in FIGS. 1 to 3 appended to the present description. FIG. 4 reports the thermogravimetric (TGA) analysis plot of the amorphous form.

New crystalline forms of the invention have been also synthesized and have been characterized by means of X-ray diffraction (XRPD), infrared (FT-IR), differential scanning calorimetry (DSC) spectra thereof, and have shown to be different from the known forms I and II above.

Characterization data of the crystalline compounds of the invention are provided in the Experimental Section of the present description and in the appended figures.

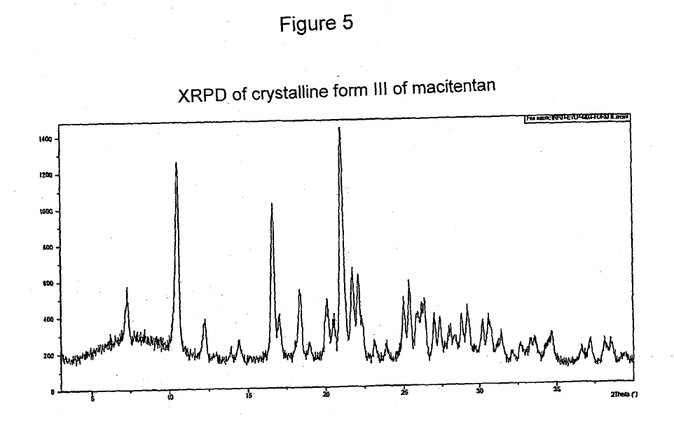

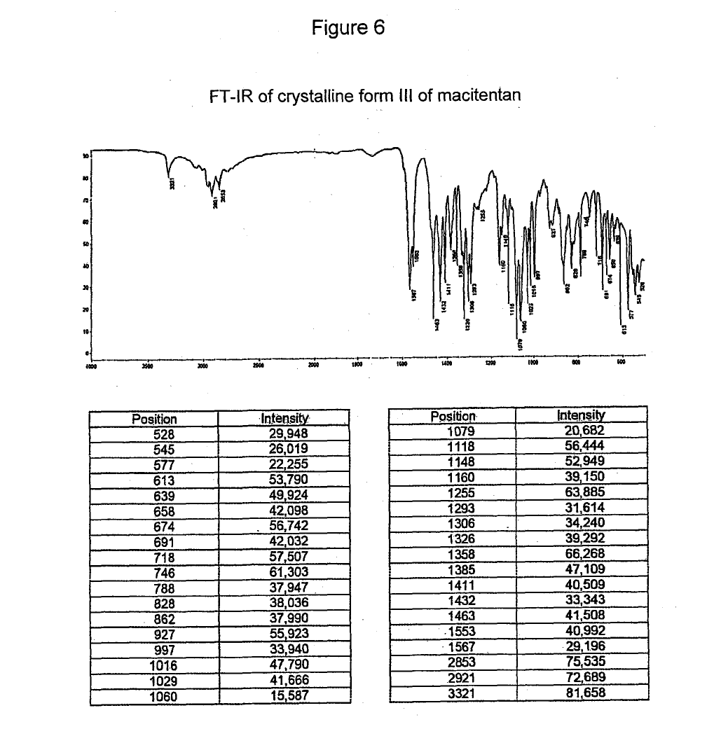

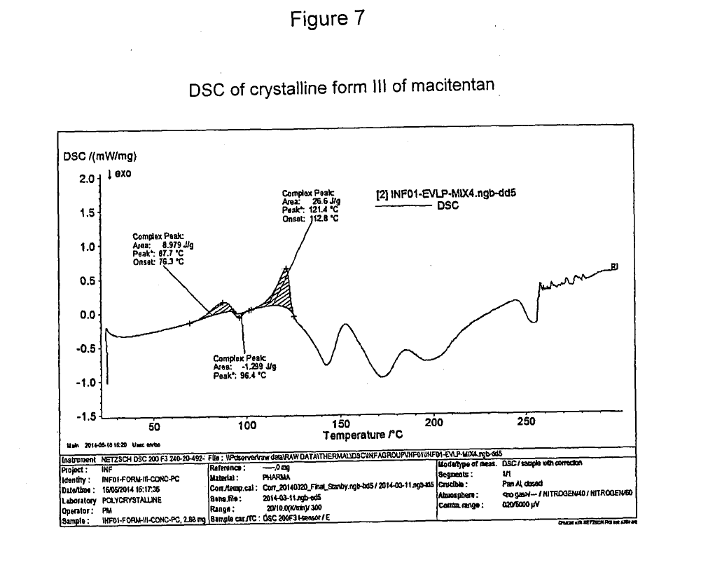

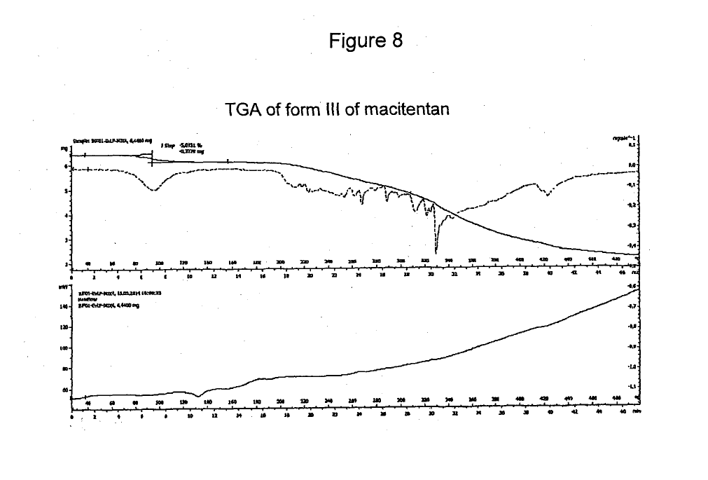

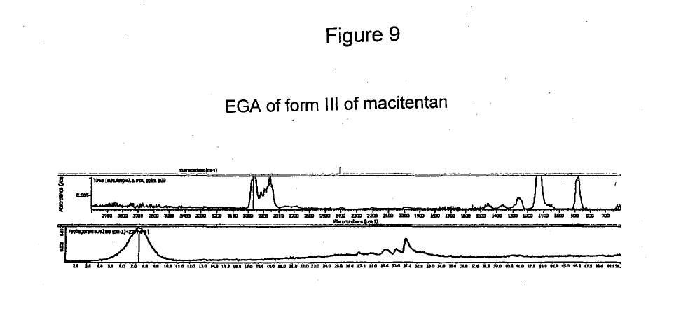

Therefore another subject-matter of the invention, according to another of the aspects thereof, is a new crystalline form of macitentan, herein defined as form III, which shows the X-ray diffraction spectrum of FIG. 5 and the IR spectrum of FIG. 6 and the DSC plot of FIG. 7. FIGS. 8 and 9 report the plots of the thermogravimetric analysis (TGA) and of the evolved gas analysis (EGA).

It has been observed that crystalline form III is a crystalline form of macitentan 1,4-dioxane solvate.

Another subject-matter of the invention, according to another of the aspects thereof, is a process for the preparation of crystalline form III, which comprises dissolving macitentan in 1,4-dioxane or in a solvent mixture comprising 1,4-dioxane and then evaporating the solvent. Advantageously, form III can be obtained from dioxane in a mixture with a solvent selected from acetonitrile, acetone, methyl ethyl ketone, chloroform and ethyl acetate. Solvent evaporation can be carried out at room temperature: 17-25° C./1 atm, at low temperature: 4-10° C./1 atm; at high temperature: 60° C./1 atm; at low pressure 17-25° C./10⁻² atm or at high temperature and low pressure: 40° C./10⁻² atm.

According to a preferred embodiment, evaporation is carried out at low temperature and at room pressure (4-10° C./1 atm) or at 40° C. and at a pressure of 10⁻² atm.

DSC profile of the crystalline form III presents a first endothermic peak at 76° C. (probably related to solvent loss) and, following an exothermic signal, a melting peak at about 113° C.

TGA and EGA analyses confirmed the presence of the solvent in the crystal.

The crystalline form III of macitentan obtained and/or obtainable with the process described above, represents a further subject-matter of the present invention.

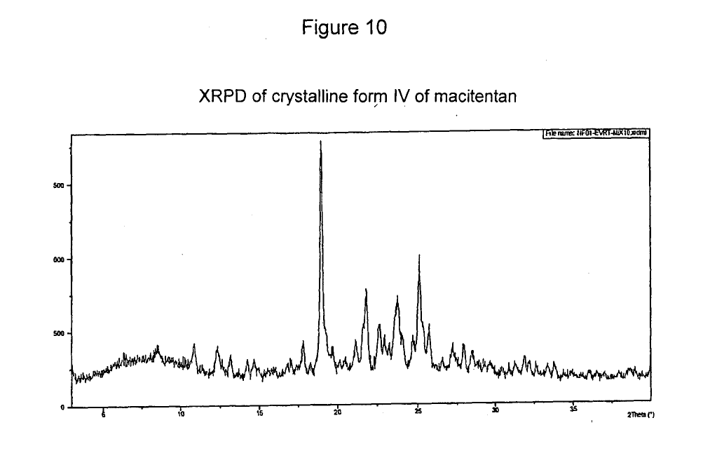

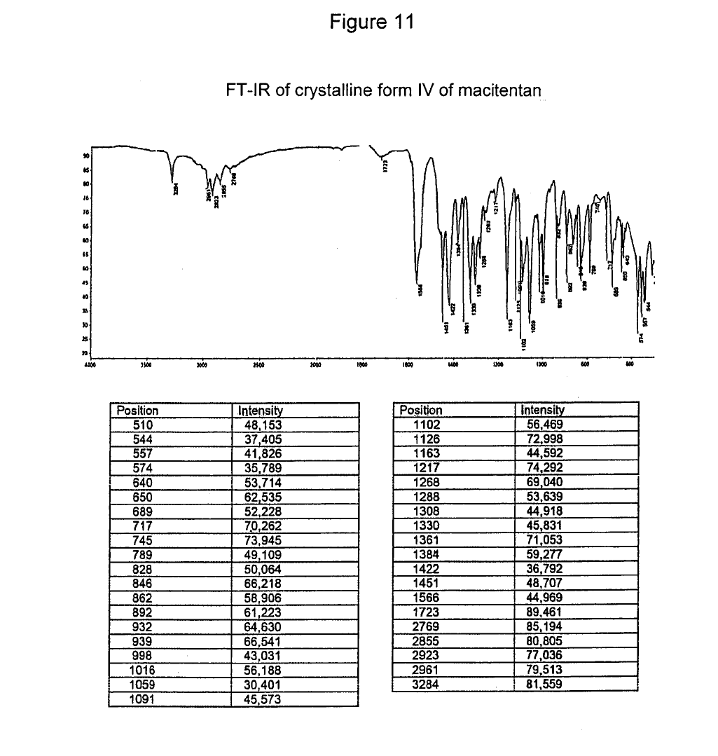

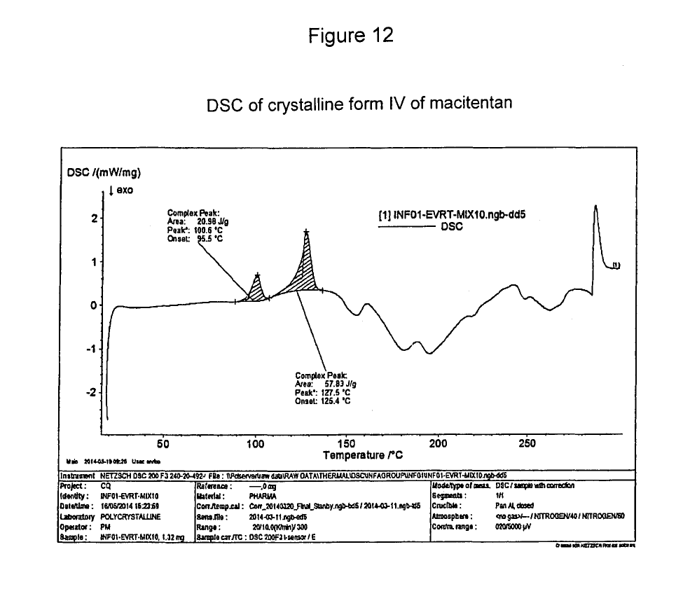

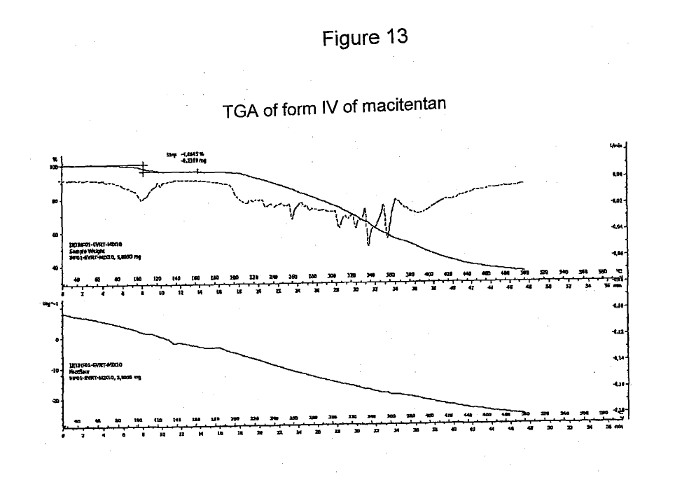

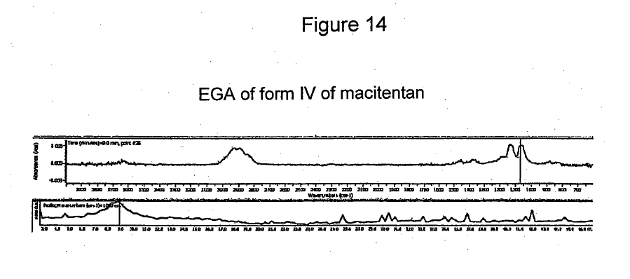

Another subject-matter of the invention, according to another of the aspects thereof, is a new crystalline form of macitentan, herein defined as form IV, which exhibits the X-ray diffraction spectrum of FIG. 10 and the FT-IR spectrum of FIG. 11 and the DSC plot of FIG. 12. FIGS. 13 and 14 report the plots of the thermogravimetric analysis (TGA) and of the evolved gas analysis (EGA).

Another subject-matter of the invention, according to another of the aspects thereof, is a process for the preparation of crystalline form IV, which comprises dissolving macitentan in a solvent mixture comprising at least 2-methoxyethanol and acetonitrile, advantageously from a mixture of 2-methoxyethanol and acetonitrile, and then evaporating the solvents.

According to a preferred embodiment, evaporation is carried out at room temperature and pressure (17-25° C./1 atm).

It has been observed that crystalline form IV is a crystalline form of macitentan 2-methoxyethanol solvate.

DSC profile of crystalline form IV has a first endothermic peak at 96° C. (probably related to solvent loss) and a melting peak at about 125° C.

TGA and EGA analyses confirmed the presence of the solvent in the crystal.

Crystalline form IV of macitentan obtained and/or obtainable with the process described above, represents a further subject-matter of the present invention.

The terms “solvate” or “solvate form” are used herein to denote that the crystalline compound comprises stoichiometric or non-stoichiometric quantities of one or more solvents.

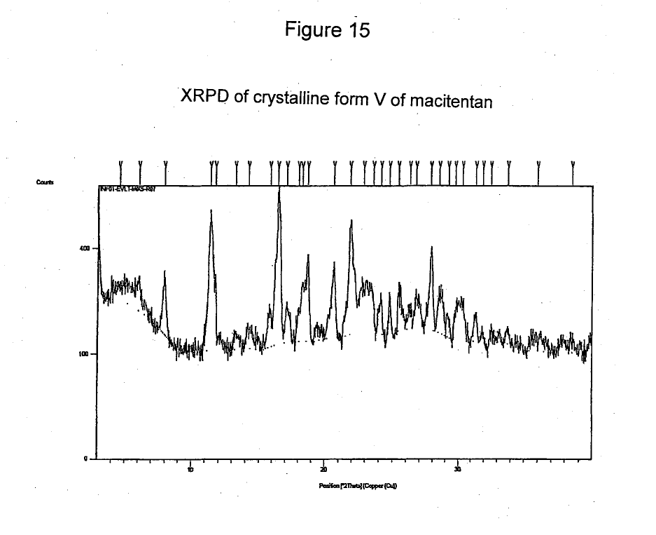

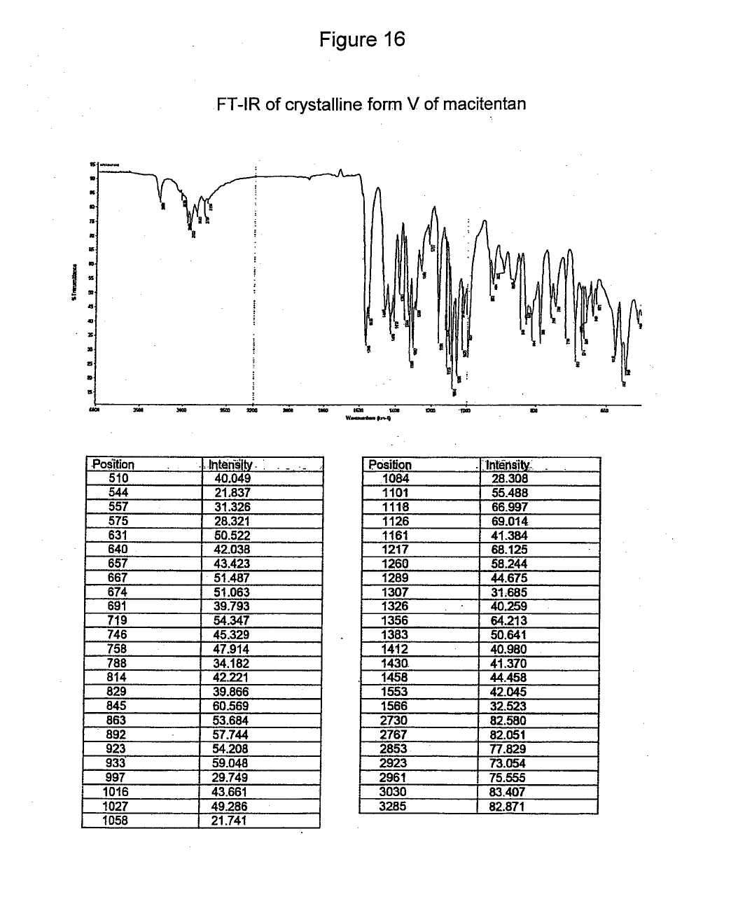

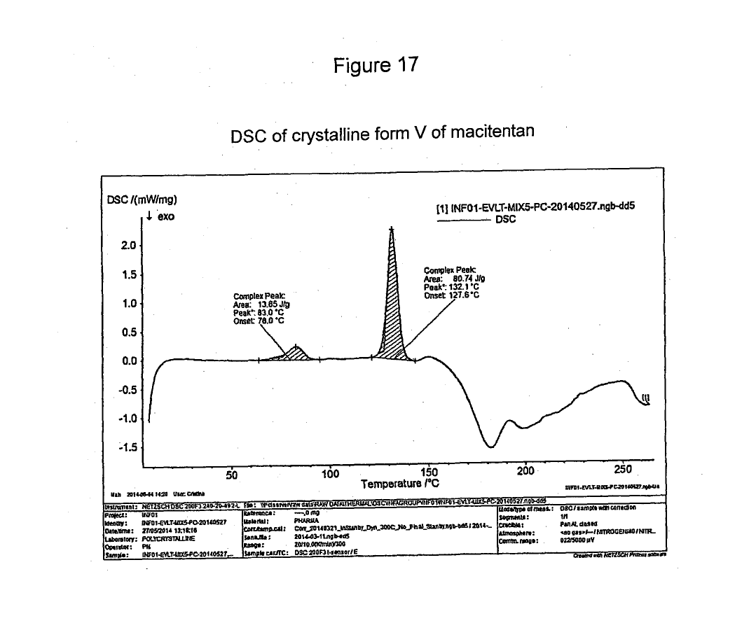

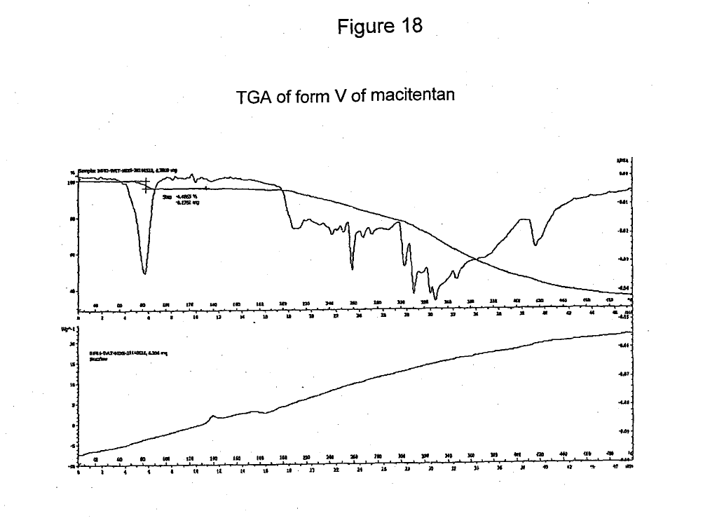

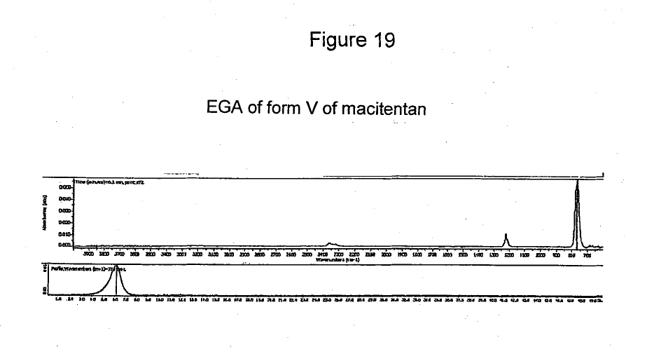

A subject-matter of the invention, according to another of the aspects thereof, is a new crystalline form of macitentan, herein defined as form V, which exhibits the X-ray diffraction spectrum of FIG. 15 and the IR spectrum of FIG. 16 and the DSC plot of FIG. 17. FIGS. 18 and 19 report the plots of the thermogravimetric analysis (TGA) and of the evolved gas analysis (EGA).

It has been observed that the crystalline form V is a crystalline form of macitentan chloroform solvate.

A subject-matter of the invention, according to another of the aspects thereof, is a process for the preparation of the crystalline form V, which comprises evaporating a solution of macitentan from a mixture of chloroform and acetone.

According to a preferred embodiment, evaporation is carried out at low temperature and room pressure.

DSC profile of crystalline form V has a first endothermic peak at 76° C. (probably related to solvent loss) and, following an exothermic signal, a melting peak at about 132° C.

TGA and EGA analyses confirmed the presence of the solvent in the crystal.

The crystalline form V of macitentan obtained and/or obtainable with the process described above, represents a further subject-matter of the present invention.

Details about the process described above are provided in the Experimental Section of the present description.

The new amorphous form of the invention showed excellent physico-chemical properties and is, therefore, a valuable alternative to the currently available macitentan forms for the administration in humans and/or animals.

A subject-matter of the invention, according to another of the aspects thereof, is a pharmaceutical composition which comprises the amorphous (non-crystalline) form of macitentan according to the invention, in combination with one or more pharmaceutically acceptable vehicles or excipients. Advantageously, the compositions of the invention comprise the amorphous form as defined in the present description and in the appended figures.

The pharmaceutical compositions of the invention are particularly suited for the oral administration.

For the oral administration, said compositions can be in the form of tablets, capsules or granules and are prepared according to conventional methods with pharmaceutically acceptable excipients such as binders, fillers, lubricants, disintegrants, wetting agents, flavors, etc. Tablets can in addition be coated by means of methods well known in the art.

The compositions of the invention are advantageously in the form of unit dose. Preferably, each unit dose according to the invention comprises 1 to 100 mg, e.g. 5 to 50 mg, advantageously 8 to 20 mg, e.g. about 10 mg, of the new crystalline forms or the amorphous form according to the invention, advantageously in combination with standard excipients and additives well known to one skilled in the field. Other dosages can be obviously provided, depending on diseases and on conditions of the subject to be treated.

According to a preferred embodiment, the compositions of the invention comprise as active ingredient, the amorphous form of macitentan, advantageously the amorphous form as defined in the present description and in the appended figures.

A subject-matter of the invention, according to another of the aspects thereof, is the amorphous form of macitentan, and/or the pharmaceutical compositions of the invention for the use thereof in the therapy, in particular in the therapy of pulmonary arterial hypertension.

The invention also comprises a method for the treatment of the pulmonary arterial hypertension which comprises administering, to a subject in need of it, an effective amount of the amorphous form of macitentan, advantageously in the form of a pharmaceutical composition as defined above.

The amorphous form and the new crystalline forms of macitentan can be also converted in other polymorphs of macitentan.

Therefore a subject-matter of the invention, according to another of the aspects thereof, is the use of the amorphous form of macitentan for the preparation of macitentan crystalline forms.

A subject-matter of the invention, according to another of the aspects thereof, is the use of a crystalline form of macitentan selected from forms (III), (IV), (V) and mixtures thereof, for the preparation of the amorphous form of macitentan or of another crystalline form of macitentan.

### Experimental Section

### XRPD

The samples underwent X-ray powder diffraction on the untreated samples.

Instrument: X'Pert PRO

| | |
|---|---|
| Scan Axis | Gonio |
| Start Position [°2Th.] | 3.0094 |
| End Position [°2Th.] | 39.9844 |
| Step Size [°2Th.] | 0.0170 |
| Scan Step Time [s] | 12.9218 |
| Scan Type | continuous |
| PSD Mode | Scan |
| PSD Length [°2Th.] | 2.12 |
| Offset [°2Th.] | 0.0000 |
| Divergence Slit Type | Fixed |
| Divergence Slit Size [°] | 0.4354 |
| Specimen Length [mm] | 10.00 |
| Measurement Temperature [° C.] | 25.00 |
| Anode Material | Cu |
| K-Alpha1 [Å] | 1.54060 |
| K-Alpha2 [Å] | 1.54443 |
| K-Beta [Å] | 1.39225 |
| K-A2/K-A1 Ratio | 0.50000 |
| Generator Settings | 40 mA, 40 kV |
| Diffractometer Type | 0000000011019590 |
| Diffractometer Number | 0 |
| Goniometer Radius [mm] | 240.00 |
| Dist. Focus-Diverg. Slit [mm] | 100.00 |
| Incident Beam Monochromator | No |
| Spinning | Yes |

### FT-IR

The analysis has been carried out using a Thermo Nicolet 6700 FT-IT spectrometer equipped with Smart performer ZnSe; DTGS Kbr Detector; IR Source; KBr Beam Splitter.

### DSC

The analysis has been carried out by using a DSC 200 F3 Maia®

| Temperature range | −170° C. . . . 600° C. |
|---|---|
| Heating rate | 0.001 K/min . . . 100 K/min |
| Cooling rate | 0.001 K/min . . . 00 K/min (depending on the |
| | temperature) |
| Sensor | “heat flux system” |
| Measurement interval | 0 mW . . . ±600 mW |
| Temperature accuracy | 0.1 K |
| Enthalpy accuracy | <1% |

### TGA

The analysis has been carried out with a Mettler Toledo Star^{e} System.

| | |
|---|---|
| Temperature interval | from room temperature to 1100° C. |
| Temperature accuracy | ±1 K |
| Temperature precision | ±0.4 K |
| Heating rate | 0.02 . . . 250 K/min |
| Cooling time | 20 min (1100 . . . 100° C.) |
| Sample volume | ≦100 μL |

### EGA

The analysis has been carried out on gases produced by the TGA.

| Automation | 34 samples positions |
|---|---|
| TGA-FTIR | coupled with a Thermo Nicolet 6700 spectrometer |
| “Balance data” | XP5 |
| Measurement range | ≦5 g |
| Resolution | 1.0 μg |
| Weight accuracy | 0.005% |
| Weight precision | 0.0025% |
| Loads of the inner ring | 2 |

Reproducibility of background curve: greater than ±10 μg over the whole temperature range

### Example 1

### Preparation of the Amorphous Form of Macitentan

A solution of 50 mg of macitentan in 5 ml of dimethyl sulfoxide (DMSO) is prepared by heating up to about 100° C. under stirring. It is then left to cool down to room temperature, filtered with a Whatman 0.45 micron filter and the solvent is evaporated at a temperature of about 60° C. and at room pressure.

The IR spectrum of the amorphous form exhibits the following absorption bands:

| Position | Intensity |
|---|---|
| 502 | 34.602 |
| 543 | 26.189 |
| 557 | 28.557 |
| 574 | 32.026 |
| 614 | 44.842 |
| 630 | 44.953 |
| 657 | 49.751 |
| 690 | 42.717 |
| 721 | 56.458 |
| 740 | 58.837 |
| 790 | 37.198 |
| 802 | 54.341 |
| 826 | 39.547 |
| 857 | 53.983 |
| 935 | 52.056 |
| 998 | 29.590 |
| 1013 | 39.744 |
| 1054 | 31.840 |
| 1083 | 35.824 |
| 1139 | 51.162 |
| 1167 | 41.863 |
| 1212 | 61.073 |
| 1305 | 33.299 |
| 1331 | 49.726 |
| 1389 | 54.374 |
| 1420 | 24.181 |
| 1452 | 51.858 |
| 1548 | 48.338 |
| 1567 | 39.023 |
| 1618 | 73.819 |
| 1652 | 76.412 |
| 2876 | 78.496 |
| 2930 | 75.954 |
| 2963 | 75.165 |
| 3048 | 79.450 |

### Example 2

### Preparation of the Crystalline Form III of Macitentan

50 mg of macitentan are dissolved in 5 ml of 1,4-dioxane. The solution is left under stirring at room temperature for about 60 minutes. It is filtered with a Whatman 0.45 micron filter and the solvent is left to evaporate. The crystalline form III of macitentan is then obtained. The X-ray diffraction spectrum showed the following characteristic peaks

| | Intensity | | | Rel. |
|---|---|---|---|---|
| Pos. [°2Th.] | [cts] | FWHM [°2Th.] | d-spacing [Å] | Int. [%] |
| 8.0493 | 435.45 | 0.1171 | 10.98421 | 15.71 |
| 11.4367 | 1661.68 | 0.1338 | 7.73737 | 59.95 |
| 13.0675 | 500.42 | 0.1171 | 6.77516 | 18.05 |
| 13.3493 | 317.58 | 0.1338 | 6.63278 | 11.46 |
| 13.9920 | 438.57 | 0.0836 | 6.32954 | 15.82 |
| 14.3374 | 428.42 | 0.1171 | 6.17780 | 15.46 |
| 16.1004 | 2771.76 | 0.1338 | 5.50509 | 100.00 |
| 17.1908 | 439.05 | 0.1338 | 5.15830 | 15.84 |
| 17.6316 | 27.22 | 0.2007 | 5.03030 | 0.98 |
| 18.1851 | 1438.57 | 0.1338 | 4.87843 | 51.90 |
| 18.5730 | 1869.92 | 0.1673 | 4.77742 | 67.46 |
| 19.6098 | 79.22 | 0.1004 | 4.52709 | 2.86 |
| 20.0937 | 705.31 | 0.1171 | 4.41915 | 25.45 |
| 20.3594 | 216.08 | 0.0836 | 4.36207 | 7.80 |
| 21.3017 | 719.95 | 0.1004 | 4.17120 | 25.97 |
| 21.4241 | 810.37 | 0.1171 | 4.14765 | 29.24 |
| 22.0501 | 809.63 | 0.1004 | 4.03129 | 29.21 |
| 22.7109 | 436.44 | 0.1004 | 3.91547 | 15.75 |
| 22.9603 | 576.61 | 0.1171 | 3.87351 | 20.80 |
| 23.4471 | 1522.65 | 0.1338 | 3.79417 | 54.93 |
| 24.5808 | 223.07 | 0.0836 | 3.62169 | 8.05 |
| 25.3961 | 1850.34 | 0.1171 | 3.50724 | 66.76 |
| 25.6632 | 252.05 | 0.1004 | 3.47134 | 9.09 |
| 26.0670 | 390.93 | 0.1004 | 3.41847 | 14.10 |
| 26.5784 | 714.24 | 0.1020 | 3.35108 | 25.77 |
| 26.6859 | 967.33 | 0.0836 | 3.34059 | 34.90 |
| 27.7052 | 328.44 | 0.0669 | 3.21996 | 11.85 |
| 28.2708 | 1010.83 | 0.1338 | 3.15681 | 36.47 |
| 28.5287 | 725.92 | 0.0836 | 3.12886 | 26.19 |
| 29.0698 | 221.25 | 0.1171 | 3.07183 | 7.98 |
| 29.4389 | 89.23 | 0.1338 | 3.03416 | 3.22 |
| 29.7298 | 134.30 | 0.1004 | 3.00512 | 4.85 |
| 30.0408 | 185.13 | 0.1171 | 2.97472 | 6.68 |
| 31.2096 | 930.92 | 0.1338 | 2.86593 | 33.59 |
| 31.7284 | 138.41 | 0.1338 | 2.82024 | 4.99 |
| 32.4998 | 215.26 | 0.0502 | 2.75505 | 7.77 |
| 32.9927 | 131.76 | 0.1338 | 2.71500 | 4.75 |
| 33.5837 | 131.91 | 0.1338 | 2.66857 | 4.76 |
| 34.3216 | 201.07 | 0.1338 | 2.61286 | 7.25 |
| 35.5495 | 164.39 | 0.2342 | 2.52538 | 5.93 |
| 36.5207 | 84.65 | 0.1338 | 2.46042 | 3.05 |
| 36.8522 | 155.73 | 0.1004 | 2.43905 | 5.62 |
| 37.3291 | 88.66 | 0.1004 | 2.40898 | 3.20 |
| 37.6369 | 238.57 | 0.1171 | 2.38998 | 8.61 |
| 38.4727 | 115.95 | 0.1338 | 2.33996 | 4.18 |
| 38.9075 | 53.88 | 0.1004 | 2.31481 | 1.94 |
| 39.6135 | 98.87 | 0.1004 | 2.27517 | 3.57 |

The IR spectrum the form III exhibits the following absorption bands:

| Position | Intensity |
|---|---|
| 528 | 29.948 |
| 545 | 26.019 |
| 577 | 22.255 |
| 613 | 53.790 |
| 639 | 49.924 |
| 658 | 42.098 |
| 674 | 56.742 |
| 691 | 42.032 |
| 718 | 57.507 |
| 746 | 61.303 |
| 788 | 37.947 |
| 828 | 38.036 |
| 862 | 37.990 |
| 927 | 55.923 |
| 997 | 33.940 |
| 1016 | 47.790 |
| 1029 | 41.666 |
| 1060 | 15.587 |
| 1079 | 20.682 |
| 1118 | 56.444 |
| 1148 | 52.949 |
| 1160 | 39.150 |
| 1255 | 63.885 |
| 1293 | 31.614 |
| 1306 | 34.240 |
| 1326 | 39.292 |
| 1358 | 66.268 |
| 1385 | 47.109 |
| 1411 | 40.509 |
| 1432 | 33.343 |
| 1463 | 41.508 |
| 1553 | 40.992 |
| 1567 | 29.196 |
| 2853 | 75.535 |
| 2921 | 72.689 |
| 3321 | 81.658 |

### Example 3

### Preparation of the Crystalline Form III of Macitentan

50 mg of macitentan are dissolved in 5 ml of a mixture of 1,4-dioxane and acetonitrile (1/1; v/v). The solution is left under stirring at room temperature for about 60 minutes. It is filtered with a Whatman 0.45 micron filter and the solvent is left to evaporate. The crystalline form III of macitentan is then obtained.

### Example 4

### Preparation of the Crystalline Form III of Macitentan

50 mg of macitentan are dissolved in 5 ml of a mixture of 1,4-dioxane and acetone (1/1; v/v). The solution is left under stirring at room temperature for about 60 minutes. It is filtered with a Whatman 0.45 micron filter and the solvent is evaporated at low pressure (10⁻² atm) and at room temperature. The crystalline form III of macitentan is then obtained.

### Example 5

### Preparation of the Crystalline Form III of Macitentan

50 mg of macitentan are dissolved in 5 ml of a mixture of 1,4-dioxane and ethanol (1/1; v/v). The solution is left under stirring at room temperature for about 60 minutes. It is filtered with a Whatman 0.45 micron filter and the solvent is evaporated at low pressure (10⁻² atm) and at 40° C. The crystalline form III of macitentan is then obtained.

### Example 6

### Preparation of the Crystalline Form III of Macitentan

50 mg of macitentan are dissolved in 5 ml of a mixture of 1,4-dioxane and chloroform (1/1; v/v). The solution is left under stirring at room temperature for about 60 minutes. It is filtered with a Whatman 0.45 micron filter and the solvent is evaporated at low temperature (about 4-10° C.) and at room pressure. The crystalline form III of macitentan is then obtained.

### Example 7

### Preparation of the Crystalline Form IV of Macitentan

50 mg of macitentan are dissolved in 5 ml of a mixture of 2-methoxyethanol/acetonitrile (1/1, v/v). The solution is left under stirring at room temperature for about 60 minutes. It is filtered with a Whatman 0.45 micron filter and the solvent is left to evaporate. The crystalline form IV of macitentan is then obtained. The X-ray diffraction spectrum showed the following characteristic peaks

| | Intensity | | | Rel. |
|---|---|---|---|---|
| Pos. [°2Th.] | [cts] | FWHM [°2Th.] | d-spacing [Å] | Int. [%] |
| 6.3286 | 56.94 | 0.2007 | 13.96646 | 3.74 |
| 8.5215 | 87.34 | 0.2007 | 10.37664 | 5.73 |
| 10.8300 | 169.10 | 0.0836 | 8.16936 | 11.09 |
| 12.2972 | 159.85 | 0.2342 | 7.19779 | 10.48 |
| 13.1878 | 112.59 | 0.2342 | 6.71362 | 7.38 |
| 14.2819 | 100.58 | 0.1338 | 6.20169 | 6.60 |
| 14.6698 | 105.34 | 0.1338 | 6.03859 | 6.91 |
| 15.7754 | 23.13 | 0.4015 | 5.61778 | 1.52 |
| 17.0345 | 59.62 | 0.1338 | 5.20527 | 3.91 |
| 17.8573 | 180.01 | 0.0836 | 4.96723 | 11.81 |
| 18.3238 | 38.74 | 0.2007 | 4.84182 | 2.54 |
| 19.0408 | 1524.57 | 0.1673 | 4.66108 | 100.00 |
| 19.7305 | 97.67 | 0.1673 | 4.49968 | 6.41 |
| 20.4674 | 49.71 | 0.1673 | 4.33932 | 3.26 |
| 21.1624 | 169.64 | 0.1673 | 4.19834 | 11.13 |
| 21.8297 | 487.97 | 0.1171 | 4.07149 | 32.01 |
| 22.6196 | 266.60 | 0.1673 | 3.93106 | 17.49 |
| 22.9343 | 175.35 | 0.1004 | 3.87783 | 11.50 |
| 23.5891 | 310.37 | 0.1338 | 3.77166 | 20.36 |
| 23.7925 | 413.62 | 0.1338 | 3.73987 | 27.13 |
| 24.0965 | 154.63 | 0.1004 | 3.69337 | 10.14 |
| 24.7001 | 190.17 | 0.1004 | 3.60446 | 12.47 |
| 25.1239 | 710.31 | 0.1171 | 3.54461 | 46.59 |
| 25.7126 | 246.95 | 0.1338 | 3.46479 | 16.20 |
| 26.5195 | 42.99 | 0.1338 | 3.36116 | 2.82 |
| 27.2119 | 123.63 | 0.2007 | 3.27719 | 8.11 |
| 27.9508 | 153.20 | 0.1673 | 3.19221 | 10.05 |
| 28.4721 | 116.55 | 0.2342 | 3.13494 | 7.64 |
| 29.6389 | 60.21 | 0.2007 | 3.01413 | 3.95 |
| 30.4165 | 21.48 | 0.2007 | 2.93883 | 1.41 |
| 30.9051 | 37.21 | 0.1004 | 2.89347 | 2.44 |
| 31.3248 | 55.82 | 0.2007 | 2.85565 | 3.66 |
| 31.8986 | 116.36 | 0.1338 | 2.80558 | 7.63 |
| 32.1976 | 68.55 | 0.1338 | 2.78021 | 4.50 |
| 32.6730 | 59.48 | 0.1338 | 2.74083 | 3.90 |
| 33.3799 | 69.90 | 0.1338 | 2.68439 | 4.58 |
| 33.7918 | 75.05 | 0.1004 | 2.65261 | 4.92 |
| 36.0388 | 36.52 | 0.2007 | 2.49220 | 2.40 |
| 37.9547 | 35.56 | 0.2007 | 2.37069 | 2.33 |
| 38.5948 | 50.19 | 0.2676 | 2.33284 | 3.29 |

The IR spectrum of form IV exhibits the following absorption bands:

| Position | Intensity |
|---|---|
| 510 | 48.153 |
| 544 | 37.405 |
| 557 | 41.826 |
| 574 | 35.789 |
| 640 | 53.714 |
| 650 | 62.535 |
| 689 | 52.228 |
| 717 | 70.262 |
| 745 | 73.945 |
| 789 | 49.109 |
| 828 | 50.064 |
| 846 | 66.218 |
| 862 | 58.906 |
| 892 | 61.223 |
| 932 | 64.630 |
| 939 | 66.541 |
| 998 | 43.031 |
| 1016 | 56.188 |
| 1059 | 30.401 |
| 1091 | 45.573 |
| 1102 | 56.469 |
| 1126 | 72.998 |
| 1163 | 44.592 |
| 1217 | 74.292 |
| 1268 | 69.040 |
| 1288 | 53.639 |
| 1308 | 44.918 |
| 1330 | 45.831 |
| 1361 | 71.053 |
| 1384 | 59.277 |
| 1422 | 36.792 |
| 1451 | 48.707 |
| 1566 | 44.969 |
| 1723 | 89.461 |
| 2769 | 85.194 |
| 2855 | 80.805 |
| 2923 | 77.036 |
| 2961 | 79.513 |
| 3284 | 81.559 |

### Example 8

### Preparation of the Crystalline Form V of Macitentan

50 mg of macitentan are dissolved in 5 ml of a mixture of chloroform and acetone (1/1, v/v) by heating to the boiling point of the mixture, under stirring. The solution is left to cool down to room temperature, it is filtered with a Whatman 0.45 micron filter and the solvent is evaporated at low temperature, about 4-10° C. and at room pressure. The crystalline form V of macitentan is then obtained. The X-ray diffraction spectrum showed the following X-ray diffraction features:

| | Intensity | | | Rel. |
|---|---|---|---|---|
| Pos. [°2Th.] | [cts] | FWHM [°2Th.] | d-spacing [Å] | Int. [%] |
| 4.5808 | 43.33 | 0.8029 | 19.29049 | 8.4 |
| 6.0557 | 97.15 | 0.2007 | 14.59529 | 18.47 |
| 7.9465 | 151.36 | 0.1673 | 11.12613 | 28.77 |
| 11.3513 | 452.23 | 0.0836 | 7.79535 | 85.97 |
| 11.7196 | 191.95 | 0.0836 | 7.55118 | 36.49 |
| 13.1926 | 32.90 | 0.3346 | 6.71119 | 6.25 |
| 14.1869 | 44.71 | 0.4015 | 6.24301 | 8.50 |
| 15.8233 | 82.44 | 0.2007 | 5.60087 | 15.67 |
| 16.4248 | 526.03 | 0.0836 | 5.39709 | 100.00 |
| 17.0945 | 94.20 | 0.1338 | 5.18714 | 17.91 |
| 17.9948 | 70.52 | 0.2007 | 4.92958 | 13.41 |
| 18.2910 | 146.63 | 0.1338 | 4.85042 | 27.87 |
| 18.7023 | 252.73 | 0.1171 | 4.74468 | 48.04 |
| 20.6271 | 215.83 | 0.1004 | 4.30606 | 41.03 |
| 21.8438 | 351.49 | 0.2007 | 4.06890 | 66.82 |
| 22.8555 | 118.34 | 0.2676 | 3.89103 | 22.50 |
| 23.5836 | 87.18 | 0.3346 | 3.77252 | 16.57 |
| 24.1386 | 91.96 | 0.1673 | 3.68703 | 17.48 |
| 24.7511 | 89.54 | 0.1338 | 3.59715 | 17.02 |
| 25.4151 | 101.97 | 0.0669 | 3.50466 | 19.38 |
| 26.2492 | 48.54 | 0.2007 | 3.39516 | 9.23 |
| 26.7233 | 107.23 | 0.1004 | 3.33600 | 20.39 |
| 27.8562 | 254.70 | 0.1004 | 3.20285 | 48.42 |
| 28.5091 | 94.58 | 0.4015 | 3.13096 | 17.98 |
| 29.1929 | 45.36 | 0.1338 | 3.05916 | 8.62 |
| 29.7448 | 61.22 | 0.3346 | 3.00365 | 11.64 |
| 30.3009 | 90.57 | 0.3346 | 2.94978 | 17.22 |
| 31.3651 | 61.97 | 0.2342 | 2.85208 | 11.78 |
| 31.8773 | 27.05 | 0.2007 | 2.80741 | 5.14 |
| 32.4984 | 21.68 | 0.2007 | 2.75516 | 4.12 |
| 33.7678 | 31.79 | 0.2342 | 2.65444 | 6.04 |
| 35.9834 | 26.40 | 0.8029 | 2.49592 | 5.02 |
| 38.5004 | 17.34 | 0.4015 | 2.33834 | 3.30 |

The IR spectrum of form V exhibits the following absorption bands:

| Position | Intensity |
|---|---|
| 510 | 40.049 |
| 544 | 21.837 |
| 557 | 31.326 |
| 575 | 28.321 |
| 631 | 50.522 |
| 640 | 42.038 |
| 657 | 43.423 |
| 667 | 51.487 |
| 674 | 51.063 |
| 691 | 39.793 |
| 719 | 54.347 |
| 746 | 45.329 |
| 758 | 47.914 |
| 788 | 34.182 |
| 814 | 42.221 |
| 829 | 39.866 |
| 845 | 60.569 |
| 863 | 53.684 |
| 892 | 57.744 |
| 923 | 54.208 |
| 933 | 59.048 |
| 997 | 29.749 |
| 1016 | 43.661 |
| 1027 | 49.286 |
| 1058 | 21.741 |
| 1084 | 28.308 |
| 1101 | 55.488 |
| 1118 | 66.997 |
| 1126 | 69.014 |
| 1161 | 41.384 |
| 1217 | 68.125 |
| 1260 | 58.244 |
| 1289 | 44.675 |
| 1307 | 31.685 |
| 1326 | 40.259 |
| 1356 | 64.213 |
| 1383 | 50.641 |
| 1412 | 40.980 |
| 1430 | 41.370 |
| 1458 | 44.458 |
| 1553 | 42.045 |
| 1566 | 32.523 |
| 2730 | 82.580 |
| 2767 | 82.051 |
| 2853 | 77.829 |
| 2923 | 73.054 |
| 2961 | 75.555 |
| 3030 | 83.407 |
| 3285 | 82.871 |

1. Amorphous form of macitentan.

2. Amorphous form of macitentan which exhibits the following absorption bands by infrared absorption spectroscopy (FT-IR):

| Position | Intensity |
|---|---|
| 502 | 34.602 |
| 543 | 26.189 |
| 557 | 28.557 |
| 574 | 32.026 |
| 614 | 44.842 |
| 630 | 44.953 |
| 657 | 49.751 |
| 690 | 42.717 |
| 721 | 56.458 |
| 740 | 58.837 |
| 790 | 37.198 |
| 802 | 54.341 |
| 826 | 39.547 |
| 857 | 53.983 |
| 935 | 52.056 |
| 998 | 29.590 |
| 1013 | 39.744 |
| 1054 | 31.840 |
| 1083 | 35.824 |
| 1139 | 51.162 |
| 1167 | 41.863 |
| 1212 | 61.073 |
| 1305 | 33.299 |
| 1331 | 49.726 |
| 1389 | 54.374 |
| 1420 | 24.181 |
| 1452 | 51.858 |
| 1548 | 48.338 |
| 1567 | 39.023 |
| 1618 | 73.819 |
| 1652 | 76.412 |
| 2876 | 78.496 |
| 2930 | 75.954 |
| 2963 | 75.165 |
| 3048 | 79.450 |

3. Process for the preparation of the amorphous form of macitentan, which comprises dissolving macitentan in dimethyl sulfoxide (DMSO) and then evaporating the solvent.

4. Amorphous form of macitentan, obtainable by the process of claim 3.

5. Pharmaceutical composition comprising the amorphous form of macitentan according to claim 1, optionally in combination with at least one pharmaceutically acceptable excipient or carrier.

6. Amorphous form of macitentan according claim 1, for use in therapy.

7. Crystalline form III of macitentan, which exhibits the X-ray diffraction spectrum of FIG. 5 and the IR spectrum of FIG. 6 and the DSC plot of FIG. 7.

8. Process for the preparation of the crystalline form III of macitentan of claim 7, which comprises dissolving macitentan in 1,4-dioxane or in a solvent mixture comprising 1,4-dioxane, and then evaporating the solvent.

9. Crystalline form III of macitentan, obtainable by the process of claim 8.

10. Crystalline form IV of macitentan, which exhibits the X-ray diffraction spectrum of FIG. 10 and the IR spectrum of FIG. 11 and the DSC plot of FIG. 12.

11. Process for the preparation of the crystalline form IV of macitentan of claim 10, which comprises dissolving macitentan in a solvent mixture comprising at least 2-methoxyethanol and acetonitrile, and then evaporating said solvents.

12. Crystalline form IV of macitentan, obtainable by the process of claim 11.

13. Crystalline form V of macitentan, which exhibits the X-ray diffraction spectrum of FIG. 15 and the IR spectrum of FIG. 16 and the DSC plot of FIG. 17.

14. Process for the preparation of the crystalline form V of macitentan of claim 13, which comprises evaporating solvents from a solution of macitentan in a mixture of chloroform and acetone.

15. Crystalline form V of macitentan, obtainable by the process of claim 14.

16. Use of crystalline forms of macitentan selected from forms (III), (IV), (V) and mixtures thereof, as starting compound for the preparation of either the amorphous form of macitentan or a crystalline form of macitentan different with respect to said starting compound.

17. Use of the amorphous form of macitentan for the preparation of a crystalline form of macitentan.
